(19) United States
(12) Patent Application Publication
(10) Pub. No.: US 2012/0097250 A1
(43) Pub. Date: Apr. 26, 2012

(54) PHOTOVOLTAIC DEVICE

(75) Inventors: Richard A. Klenkler, Oakville (CA); Avery P. Yuen, Mississauga (CA); Nathan M. Bamsey, Burlington (CA)

(73) Assignee: XEROX CORPORATION, Norwalk, CT (US)

(21) Appl. No.: 12/910,028

(22) Filed: Oct. 22, 2010

(51) Int. Cl. H01L 31/0256

(52) U.S. Cl. 136/263

(57) ABSTRACT

A photovoltaic device is disclosed. The photovoltaic device includes a substrate, an anode, a cathode, and two semiconducting layers. The first semiconducting layer comprises a phthalocyanine. The second semiconducting layer includes a blend of a polythiophene with an electron acceptor. The complementary absorption profiles of these layers result in a device having greater absorption and efficiency.

### BACKGROUND

The present disclosure relates to a photovoltaic device useful for generating an electrical current upon exposure to wide spectrum light, such as sunlight. The materials described herein can be used in organic solar cells.

A photovoltaic device typically contains a layer of a photoactive material sandwiched between two electrodes (i.e. a cathode and an anode). The photoactive layer can absorb the energy in a photon emitted by radiation, such as sunlight. This photon energy creates an exciton, or bound electron-hole pair. Depending on the material, the electron and hole can travel a short distance (on the order of several nanometers) before spontaneous recombination occurs. The exciton can move to a junction where they can be separated, so that electrons are collected at one electrode and holes are collected at the other electrode. This allows current to flow through an external circuit.

Such light absorption and charge generation is limited in organic photovoltaic devices. Organic semiconducting materials arouse interest due to their low-cost potential, light weight, and ease of processing. However, the materials typically used in organic solar cells do not optimally match the solar spectrum, resulting in a large fraction of the light energy passing through the device being lost (i.e. not converted into electrical current) and low power conversion efficiency. With over half of the total solar irradiance residing in wavelengths above 650 nm, capturing longer wavelengths in this near infrared (NIR) range of from about 650 nm to about 1000 nm is desirable.

One highly studied group of materials is that of metallophthalocyanines, which are a small molecule containing a metal atom at the center of a cyclic molecule. Metallophthalocyanines generally have a high absorption coefficient (α>10⁵ cm⁻¹) and hole mobilities of around 10⁻³ cm²/V·sec. They typically have a Q-band peak in the red to near-infrared wavelengths. However, they also have a relatively narrow absorption profile.

It would be desirable to provide a photovoltaic device that can capture more of the light energy present in sunlight and generate greater amounts of electricity, increasing the power conversion efficiency of the device.

### BRIEF DESCRIPTION

Disclosed in various embodiments herein are photovoltaic devices that have an improved overall power conversion efficiency (PCE). Generally speaking, the photovoltaic devices include two semiconducting layers. The first layer contains a phthalocyanine. The second layer contains a blend of a polythiophene and an electron acceptor. The first layer is proximal to the anode, and the second layer is proximal to the cathode.

Disclosed in further embodiments is a photovoltaic device, comprising: a substrate; an anode upon the substrate; a first semiconducting layer comprising a phthalocyanine; a second semiconducting layer comprising a polythiophene and an electron acceptor; and a cathode. The first and second semiconducting layers are located between the anode and the cathode. The first semiconducting layer is located closer in distance to the anode than the second semiconducting layer. The polythiophene has the structure of Formula (II):

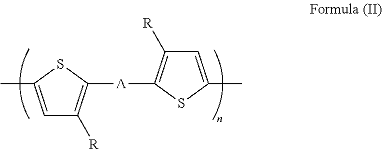

wherein A is a divalent linkage; each R is independently selected from hydrogen, alkyl, substituted alkyl, aryl, substituted aryl, alkoxy or substituted alkoxy, a heteroatom-containing group, halogen, —CN, or —NO₂; and n is from 2 to about 5,000.

The phthalocyanine may be of Formula (I):

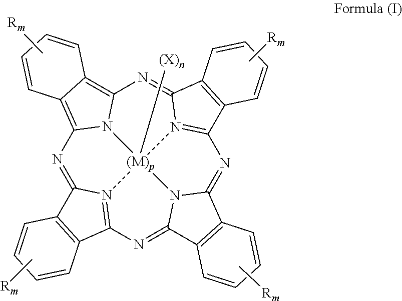

wherein M is a divalent, trivalent, or tetravalent metal atom; X is hydroxyl or halogen, and n is an integer from 0 to 2, or (X)ₙ is ═O; each m represents the number of R substituents on the phenyl ring, and is independently an integer from 0 to 6; each R is independently selected from the group consisting of halogen, alkyl, substituted alkyl, alkoxy, substituted alkoxy, phenoxy, phenylthio, aryl, substituted aryl, heteroaryl, —CN, and —NO₂; and p is 0 or 1.

In particular embodiments, the phthalocyanine is titanium oxide phthalocyanine, indium chloride phthalocyanine, dihydrogen phthalocyanine, or copper phthalocyanine.

In specific embodiments, the polythiophene is of Formula (III):

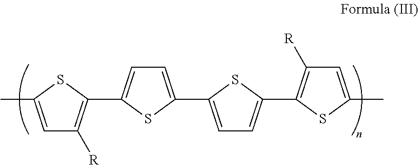

wherein R is alkyl.

In particular embodiments, the polythiophene is known as PQT-12 and has the structure of Formula (8):

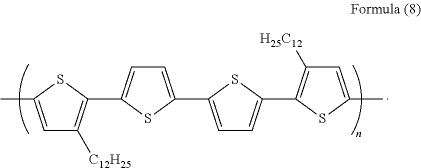

The weight ratio of the polythiophene to the electron acceptor may be from 1:99 to 99:1.

The electron acceptor may be C₆₀ fullerene, [6,6]-phenyl-C₆₁-butyric acid methyl ester (PCBM), C₇₀ fullerene, [6,6]-phenyl-C₇₁-butyric acid methyl ester, or a fullerene derivative. In particular embodiments, the electron acceptor is PCBM. In specific embodiments, the second semiconducting layer is a blend of PQT-12 and PCBM.

The first electrode may comprise indium tin oxide, fluorine tin oxide, doped zinc oxide, poly(3,4-ethylenedioxythiophene):poly(styrene sulfonic acid) (PEDOT:PSS), carbon nanotube, or graphene.

The cathode may comprise aluminum, calcium, silver, magnesium, or alloys thereof.

The photovoltaic device may additionally comprise a hole blocking layer located between the second semiconducting layer and the second electrode. The hole blocking layer may comprise bathocuproine, lithium fluoride, or bathophenanthroline.

The photovoltaic device may further comprise an electron blocking layer between the first electrode and the first semiconducting layer. The electron blocking layer may comprise poly(3,4-ethylenedioxythiophene):poly(styrene sulfonic acid) (PEDOT:PSS), MoO₃, or V₂O₅.

Also disclosed is a photovoltaic device, comprising in sequence: a substrate; an anode upon the substrate; an electron blocking layer; a first semiconducting layer comprising a phthalocyanine; a second semiconducting layer comprising a polythiophene and [6,6]-phenyl-C₆₁-butyric acid methyl ester (PCBM); and a cathode upon the second semiconducting layer. The polythiophene has the structure of Formula (II):

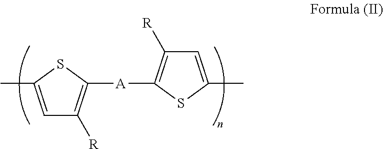

wherein A is a divalent linkage; each R is independently selected from hydrogen, alkyl, substituted alkyl, aryl, substituted aryl, alkoxy or substituted alkoxy, a heteroatom-containing group, halogen, —CN, or —NO₂; and n is from 2 to about 5,000.

Also disclosed is a photovoltaic device, comprising in sequence: an optically transparent substrate; an indium tin oxide electrode upon the substrate; an electron blocking layer comprising poly(3,4-ethylenedioxythiophene):poly(styrene sulfonic acid) (PEDOT:PSS); a first semiconducting layer comprising oxytitanium phthalocyanine; a second semiconducting layer comprising poly(3,3′″-didodecylquaterthiophene) and [6,6]-phenyl-C₆₁-butyric acid methyl ester (PCBM); and an aluminum electrode deposited on the second semiconducting layer.

These and other non-limiting aspects of the present disclosure are more particularly described below.

### BRIEF DESCRIPTION OF THE DRAWINGS

The following is a brief description of the drawings, which are presented for the purpose of illustrating the exemplary embodiments disclosed herein and not for the purpose of limiting the same.

FIG. 1 is a cross-sectional view of a first exemplary photovoltaic device of the present disclosure.

FIG. 2 is a cross-sectional view of a second exemplary photovoltaic device of the present disclosure.

FIG. 3 is a graph showing the current density versus the applied voltage for eight different photovoltaic devices of the present disclosure.

FIG. 4 is a graph showing the external quantum efficiency for one device of the present disclosure against a control, and the absorption versus the wavelength for three thin films.

### DETAILED DESCRIPTION

A more complete understanding of the components, processes, and apparatuses disclosed herein can be obtained by reference to the accompanying figures. These figures are merely schematic representations based on convenience and the ease of demonstrating the present development and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The modifier “about” used in connection with a quantity is inclusive of the stated value and has the meaning dictated by the context (for example, it includes at least the degree of error associated with the measurement of the particular quantity). When used in the context of a range, the modifier “about” should also be considered as disclosing the range defined by the absolute values of the two endpoints. For example, the range “from about 2 to about 4” also discloses the range “from 2 to 4.”

The term “comprising” is used herein as requiring the presence of the named component and allowing the presence of other components. The term “comprising” should be construed to include the term “consisting of”, which allows the presence of only the named component, along with any impurities that might result from the manufacture of the named component.

The term “upon” as used herein should be construed as describing the location of a first component relative to the second component. The term should not be construed as requiring that the first component directly contact the second component, although this direct contact is covered by the use of the term.

The present disclosure relates to a photovoltaic device containing two semiconducting layers. One of the layers includes a phthalocyanine, and the other sublayer includes a blend of a polythiophene with an electron acceptor. The phthalocyanine and polythiophene generally have complementary absorption profiles.

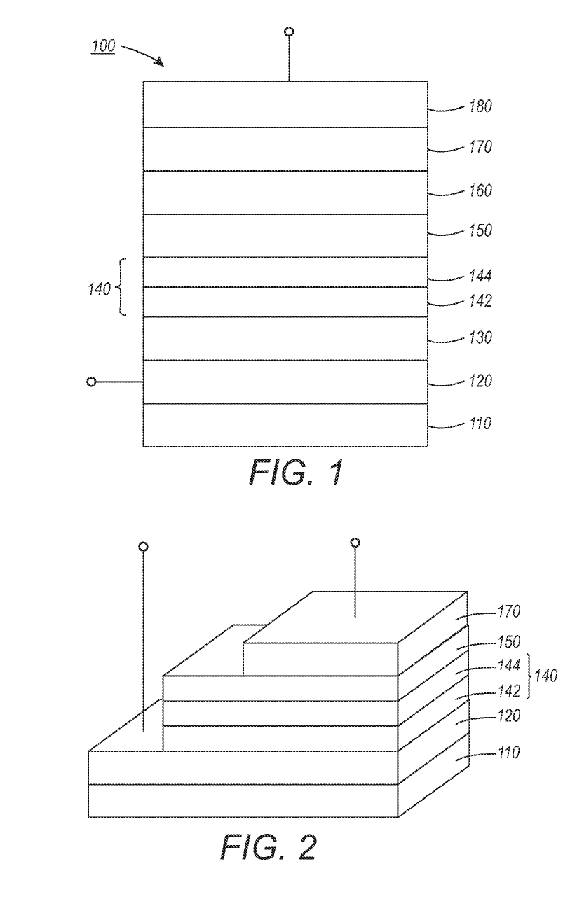

FIG. 1 is a side cross-sectional view of an exemplary photovoltaic device 100. A substrate 110 is provided. A first electrode, such as anode 120, is located upon the substrate 110. A first semiconducting layer 140 is then located upon the anode 120. As will be described further herein, the semiconducting bilayer 140 may include a first sublayer 142 and a second sublayer 144. A second semiconducting layer 150 contacts the first semiconducting layer 140. The first semiconducting layer 140 is located closer in distance to the anode 120 than the second semiconducting layer 150. An optional electron blocking layer 130 may be located between the anode 120 and the first semiconducting layer 140, if desired. An electron transporting layer 160 contacts the second semiconducting layer 150. An optional hole blocking layer 170 is located on the electron transporting layer 160. Finally, a second electrode, such as cathode 180, is placed upon the substrate 110 and on the hole blocking layer 170. The second semiconducting layer 150 is closer to the cathode 180 than the first semiconducting layer 140 is. It should also be noted that the anode 120 is closer to the substrate 110 than the cathode 180.

As illustrated in FIG. 2, only the substrate 110, anode 120, first semiconducting layer 140, second semiconducting layer 150, and cathode 180 are required to produce a functioning photovoltaic device. However, the additional layers are also helpful in obtaining a highly efficient photovoltaic device. When described in other terms, the first semiconducting layer 140 and the second semiconducting layer 150 are located between the anode 120 and the cathode 180. Also, the electron transporting layer 160 is located between the second semiconducting layer 150 and the cathode 180. The hole blocking layer 170 is located between the second semiconducting layer 150 and the cathode 180 as well. When both an electron transporting layer and hole blocking layer are present, the hole blocking layer 170 is located between the electron transporting layer 160 and the cathode 180.

The first semiconducting layer 140 contains a phthalocyanine. Phthalocyanines are hole transport molecules, completely conjugated, and have exceptional stability and color fastness. Their structure allows bonded species to protrude from the plane, modifying the packing and crystal structure. They generally have high absorption coefficients (α>10⁵ cm⁻¹) at peak absorbance. They also have strong photoelectrical properties in the NIR range, making them useful in a photovoltaic device. These phthalocyanines can be considered as photon absorbers and electron donors. It should be noted that metallophthalocyanines do not include subphthalocyanines, which have only three benzene rings, whereas metallphthalocyanines have four benzene rings in their structure.

In embodiments, the phthalocyanine is of Formula (I):

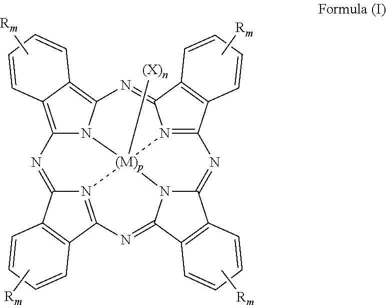

wherein M is a divalent, trivalent, or tetravalent metal atom; X is hydroxyl or halogen, and n is an integer from 0 to 2, or (X)ₙ is ═O (i.e. a double-bonded oxygen atom, also referred to as “oxo”); each m represents the number of R substituents on the phenyl ring, and is independently an integer from 0 to 6; each R is independently selected from the group consisting of halogen, alkyl, substituted alkyl, alkoxy, substituted alkoxy, phenoxy, phenylthio, aryl, substituted aryl, heteroaryl, —CN, and —NO₂; and p is 0 or 1.

The term “alkyl” refers to a radical composed entirely of carbon atoms and hydrogen atoms which is fully saturated and of the formula CₙH₂ₙ₊₁. The alkyl radical may be linear, branched, or cyclic.

The term “alkoxy” refers to an alkyl radical which is attached to an oxygen atom, i.e. —O—CₙH₂ₙ₊₁.

The term “aryl” refers to an aromatic radical composed entirely of carbon atoms and hydrogen atoms. When aryl is described in connection with a numerical range of carbon atoms, it should not be construed as including substituted aromatic radicals. For example, the phrase “aryl containing from 6 to 10 carbon atoms” should be construed as referring to a phenyl group (6 carbon atoms) or a naphthyl group (10 carbon atoms) only, and should not be construed as including a methylphenyl group (7 carbon atoms).

The term “heteroaryl” refers to an aromatic radical containing at least one heteroatom replacing a carbon atom in the radical. The heteroatom is generally nitrogen, oxygen, or sulfur.

The term “substituted” refers to at least one hydrogen atom on the named radical being substituted with another functional group, such as halogen, —CN, —NO₂, —COOH, and —SO₃H. An exemplary substituted alkyl group is a perhaloalkyl group, wherein one or more hydrogen atoms in an alkyl group are replaced with halogen atoms, such as fluorine, chlorine, iodine, and bromine.

Generally, the alkyl and alkoxy groups each independently contain from 1 to 30 carbon atoms. Similarly, the aryl groups independently contain from 6 to 30 carbon atoms.

In certain embodiments, the divalent metal atom M may be selected from the group consisting of copper, zinc, magnesium, tin, lead, nickel, cobalt, antimony, iron, and manganese. The trivalent metal atom M may be selected from the group consisting of indium(III), gallium(III), and aluminum(III). The tetravalent metal atom M may be selected from the group consisting of vanadium(IV) and titanium(IV).

Exemplary phthalocyanines include indium chloride phthalocyanine (ClInPc), aluminum chloride phthalocyanine (ClAlPc), gallium chloride phthalocyanine (ClGaPc), vanadium oxide phthalocyanine (VOPc), titanium oxide phthalocyanine (TiOPc), and copper phthalocyanine (CuPc). When p is 0, the compound is dihydrogen phthalocyanine (H₂Pc). These phthalocyanines are illustrated here as Formulas (1)-(7).

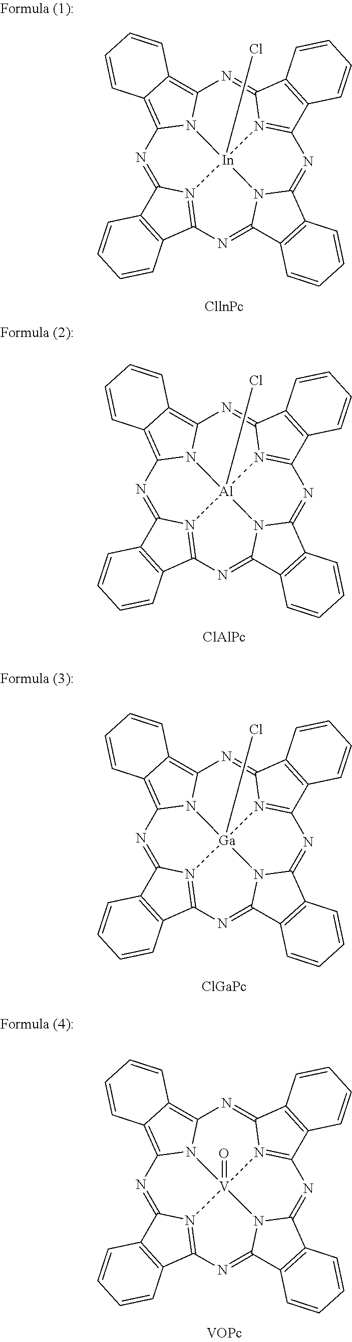

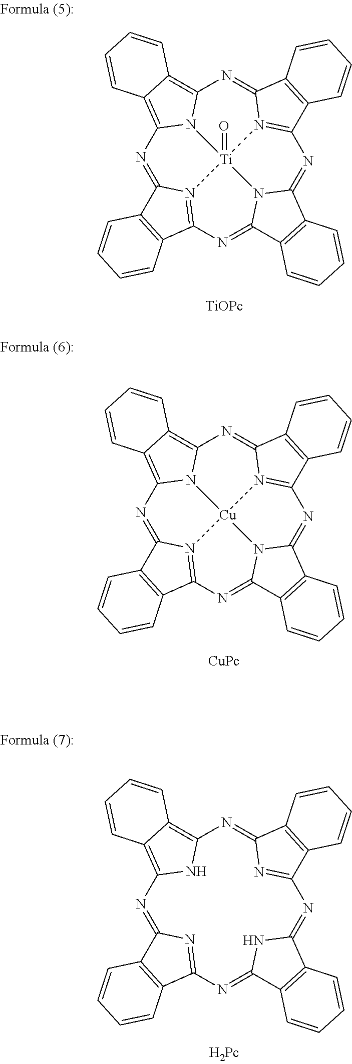

In particular embodiments, the phthalocyanine is oxytitanium phthalocyanine, dihydrogen phthalocyanine, or indium chloride phthalocyanine. In experiments, these three phthalocyanines provided an unexpectedly high improvement in photoelectrical properties. In other embodiments, the phthalocyanine is a metallophthalocyanine, where p=1.

The second semiconducting layer 150 comprises a polythiophene and an electron acceptor. An electron acceptor is a material or compound that accepts electrons transferred to it by another compound. Generally speaking, the electron acceptor moves electrons more efficiently than the polythiophene. Exemplary materials that can be used as the electron acceptor include C₆₀ fullerene, [6,6]-phenyl-C₆₁-butyric acid methyl ester (PCBM), C₇₀ fullerene, [6,6]-phenyl-C₇₁-butyric acid methyl ester (PC[70]BM), or any fullerene derivative. In particular embodiments, the electron acceptor is PCBM. The first semiconducting layer does not include an electron acceptor. PCBM has the following formula:

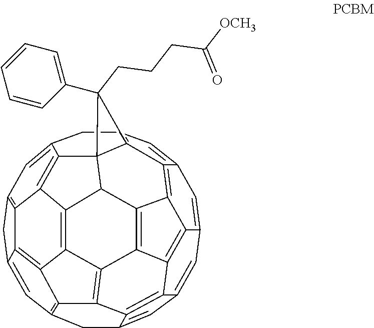

C₇₀ fullerene and PC[70]BM have the following formulas:

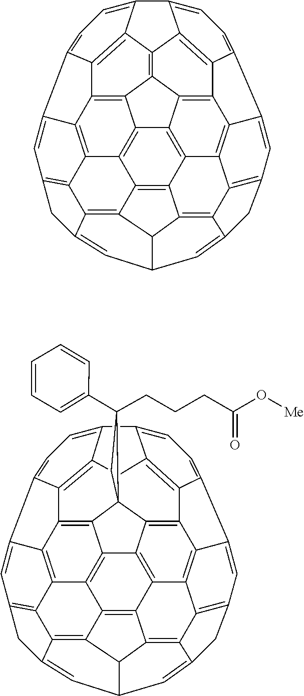

In embodiments, the polythiophene is of Formula (II):

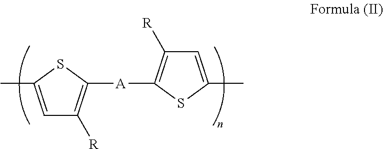

wherein A is a divalent linkage; each R is independently selected from hydrogen, alkyl, substituted alkyl, aryl, substituted aryl, alkoxy or substituted alkoxy, a heteroatom-containing group, halogen, —CN, or —NO₂; and n is from 2 to about 5,000.

The term “heteroatom-containing group” refers to a radical which is originally composed of carbon atoms and hydrogen atoms and forms a linear, branched, or cyclic backbone. This original radical can be saturated or unsaturated. One or more of the carbon atoms in the backbone is then replaced by a heteroatom, generally nitrogen, oxygen, or sulfur, to obtain a heteroatom-containing group. Exemplary heteroatom-containing groups include pyridinyl (—C₅H₅N) or furyl (—C₄H₄O).

The divalent linkage A forms a single bond to each of the two thienyl moieties in Formula (II). Exemplary divalent linkages A include:

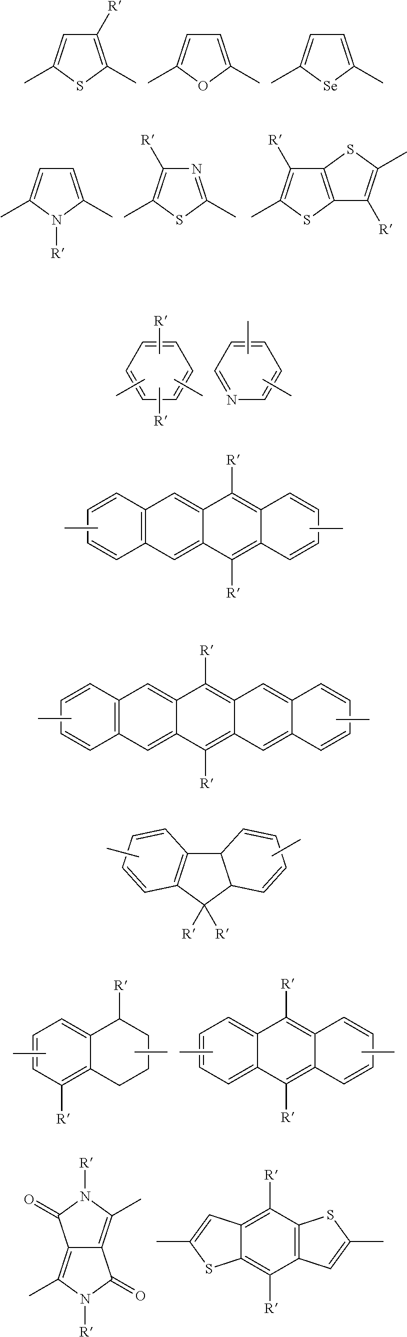

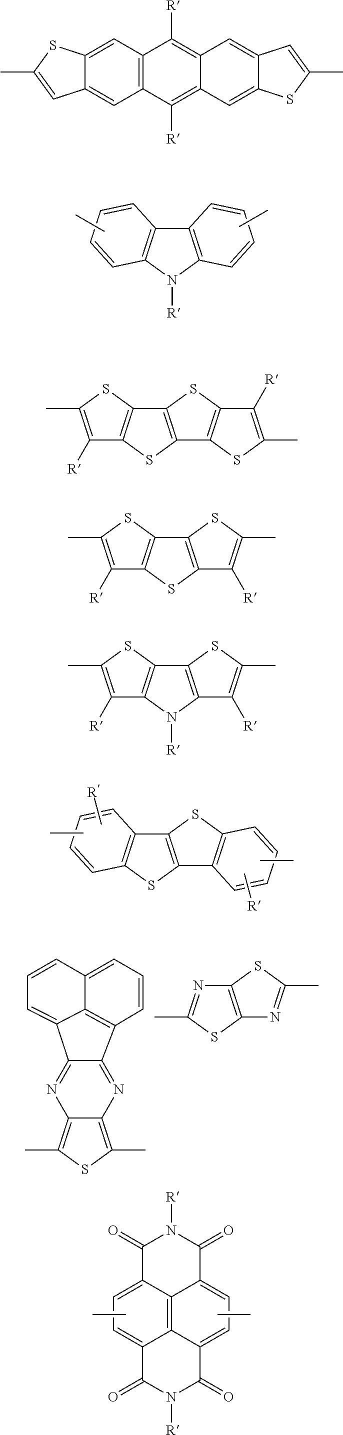

and combinations thereof, wherein each R′ is independently selected from hydrogen, alkyl, substituted alkyl, aryl, substituted aryl, alkoxy or substituted alkoxy, a heteroatom-containing group, halogen, —CN, or —NO₂. One or more of these moieties may be present in divalent linkage A. In addition, one or more of a particular moiety may be present in divalent linkage A.

It should be noted that the divalent linkage A will always be different from the two thiophene monomers shown in Formula (II); in other words, Formula (II) will not reduce to being a polythiophene made from only one monomer. In particular embodiments, A is a thienyl moiety which is different from that of the two thiophene monomers shown in Formula (II).

In more specific embodiments, the polythiophene in the second semiconducting layer is of Formula (III):

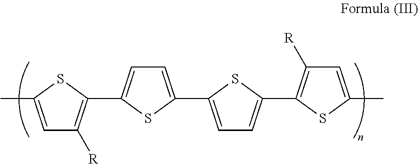

wherein R is alkyl. As discussed above, R may have from 1 to 30 carbon atoms.

In specific embodiments, the polythiophene in the second semiconducting layer is of Formula (8):

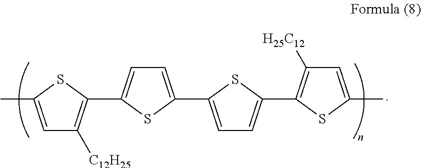

The polythiophene of Formula (8) has the name poly(3,3′″-didodecylquaterthiophene), or PQT-12.

In the second semiconducting layer, the weight ratio of the polythiophene to PCBM is from 1:99 to 99:1, based on the weight of the polythiophene and the PCBM. In some embodiments, the weight ratio is from 10:90 to 30:70. Desirably, the second semiconducting layer is a homogeneous blend of the polythiophene and the electron acceptor, although some separation of the two components may occur in isolated portions of the second layer.

In this regard, the absorption profiles of the two semiconducting layers complement each other, resulting in improved current generation. Most light absorption by the polythiophene:electron acceptor blend occurs below 650 nm, while the phthalocyanine absorbs light at wavelengths of about 650 nm to about 900 nm. Free charges created at the junction of the first and second semiconducting layers can travel through the electron acceptor to the cathode.

The first semiconducting layer (containing the phthalocyanine) has a thickness of at least 3 nanometers. In the case of a thin film (approximately 2 nm or less), the film may aggregate into isolated crystallites, leaving holes in the film. This is undesirable. It is contemplated that the first semiconducting layer is a continuous film. Put another way, the second semiconducting layer does not contact the component of the device that is on the other side of the first semiconducting layer. The second semiconducting layer (containing the blend of the polythiophene and the electron acceptor) has a thickness of at least 10 nanometers.

The first semiconducting layer, containing the phthalocyanine, is typically deposited using vacuum physical vapor deposition, which is a common industrial thin-film fabrication technique. Other deposition techniques can include liquid deposition, such as spin coating, dip coating, blade coating, rod coating, screen printing, stamping, and ink jet printing, as well as other conventional processes known in the art.

If desired, a chemical treatment can be applied to the first semiconducting layer to change the polymorph of the originally-deposited phthalocyanine. A polymorph is a specific crystalline structure of the phthalocyanine, and phthalocyanines may have multiple crystal structures, or in other words more than one polymorphic form. Several different metallophthalocyanines are known to undergo polymorphic changes when chemically treated. Several different chemical treatments can be used to change the metallophthalocyanine from one polymorph to another polymorph. One method is by solvent treatment. Solvent vapor exposure, for example to vapors of tetrahydrofuran (THF), has been shown to modify the structure and properties of several moieties of metallophthalocyanines. Similarly, several metallophthalocyanines are easily converted to different polymorphs. Solvent allows swelling and relaxation of the metallophthalocyanine film, resulting in highly photosensitive and dimorphic structures. This also extends the absorption profile of some polymorphs beyond 900 nm. Another method is thermal treatment. Metallophthalocyanines can undergo similar polymorphic changes upon heating. The presence of a different polymorph of the phthalocyanine in the first semiconducting layer can be confirmed by techniques including X-ray diffraction (XRD) and other means known in the art.

The second semiconducting layer is generally formed from a liquid composition(s), such as a dispersion or solution. The liquid composition is made by dissolving the polythiophene and the electron acceptor in an organic solvent. Exemplary solvents may include methylene chloride, tetrahydrofuran, toluene, xylene, mesitylene, chlorobenzene, or dichlorobenzene. The liquid composition is then deposited onto the device using deposition methods such as spin coating, dip coating, blade coating, rod coating, screen printing, offset printing, stamping, ink jet printing, and the like, and other conventional processes known in the art.

One structure of organic solar cells that has been explored to increase efficiency has been a series tandem cell, where layers having different absorption characteristics are stacked on top of each other and connected via a recombination layer. The recombination layer will absorb and reflect light, decreasing the amount of transmitted light available for absorption in one layer. In addition, the short circuit current density (J_{SC}) of the overall device is the lowest J_{SC} of each individual absorption layer. Thus, the short circuit current density (J_{SC}) of each layer is usually tuned to match. Because the current is heavily dependent on the thickness and structure of these layers (much more so than the voltage), the manufacturing process for a series tandem cell is much more difficult, because small changes in thickness or structure can lead to such wide variability in device performance.

In contrast, the parallel tandem cell of the present disclosure does not require a complicated recombination layer, and does not require the J_{SC} of each layer to be matched. However, the absorption profile of the parallel tandem cell captures just as wide a portion of the solar spectrum as a traditional series tandem cell.

The substrate 110 of the photovoltaic device supports the other components of the photovoltaic device. The substrate should also be optically transparent in at least the NIR range of the spectrum, to allow light to pass through and contact the semiconducting bilayer. In embodiments, the substrate is composed of materials including, but not limited to, glass, silicon, or a plastic film or sheet. For structurally flexible devices, plastic substrate, such as for example polyester, polycarbonate, polyimide sheets and the like may be used. The thickness of the substrate may be from about 10 micrometers to over 10 millimeters with an exemplary thickness being from about 50 micrometers to about 5 millimeters, especially for a flexible plastic substrate and from about 0.5 to about 10 millimeters for a rigid substrate such as glass or silicon.

The anode 120 and the cathode 180 are composed of an electrically conductive material. Exemplary materials suitable for the electrodes include aluminum, gold, silver, chromium, nickel, platinum, indium tin oxide (ITO), zinc oxide (ZnO), and the like. One of the electrodes, and in particular the anode, is made of an optically transparent material like ITO or ZnO. In specific embodiments, the anode is ITO and the cathode is aluminum. Typical thicknesses for the electrodes are about, for example, from about 40 nanometers to about 1 micrometer, with a more specific thickness being about 40 to about 400 nanometers.

An electron blocking layer 130 may be present between the anode 120 and the first semiconducting layer 140. This layer prevents recombination at the anode by inhibiting the movement of electrons to the anode. Exemplary materials include poly(3,4-ethylenedioxythiophene):poly(styrene sulfonic acid) (PEDOT:PSS), MoO₃, and V₂O₅. The electron blocking layer may have a thickness of from about 1 to about 100 nanometers.

An electron transporting layer 160 may be present between the second semiconducting layer 150 and the cathode 180. This layer is generally made from a material which allows electrons to move efficiently, and may also absorb some light wavelengths. Exemplary materials for the electron transporting layer include C₆₀ fullerene, [6,6]-phenyl-C₆₁-butyric acid methyl ester (PCBM), C₇₀ fullerene, [6,6]-phenyl-C₇₁-butyric acid methyl ester (PC[70]BM), or any fullerene derivative. The electron transporting layer may have a thickness of from about 1 nanometers to about 50 nanometers.

A hole blocking layer 170 may also be located between the second semiconducting layer 150 and the cathode 180. When the electron transporting layer is present, the hole blocking layer 170 is between the electron transporting layer 160 and the cathode 180. Exemplary hole blocking materials for this layer include bathocuproine (BCP), lithium fluoride, and bathophenanthroline. The hole blocking layer may have a thickness of from about 0.1 nanometers to about 100 nanometers.

The following examples illustrate organic photovoltaic devices made according to the methods of the present disclosure. The examples are merely illustrative and are not intended to limit the present disclosure with regard to the materials, conditions, or process parameters set forth therein. All parts are percentages by weight unless otherwise indicated.

### EXAMPLES

### Device Fabrication Procedure

An indium tin oxide (ITO) coated aluminosilicate glass substrate (50 mm×50 mm) was provided. The ITO was present in an amount sufficient to achieve a sheet resistance of 15 Ω/sq, and acted as an anode. The substrate cleaning procedure included washing with soap solution, de-ionized water, methanol, isopropanol, and then UV-ozone exposure.

Water soluble PEDOT:PSS was spun onto the cleaned substrate at 1000 rpm to form an electron blocking layer having a thickness of about 30 nm. The substrate was then annealed at 120° C. for 20 minutes in a glovebox having low O₂ (<2.0%) and low humidity (<1.0%).

The substrate was transferred to a cryopump equipped thermal evaporator, and a vacuum of less than 5×10⁻⁴ Pa was reached before material evaporation commenced. A first semiconducting layer of phthalocyanine was deposited by vacuum physical vapor deposition. The semiconducting layer had a thickness of 16 nanometers. Quartz crystal monitors were used to control the layer thickness.

The substrate was transferred back to the glovebox. A 20 mg/ml solution solution of 15 wt % PQT-12 and 85 wt % PCBM dissolved in 1,2-dichlorobenzene was spun at 1000 rpm for 60 seconds to form a second semiconducting layer approximately 50 nm thick. An aluminum cathode was evaporated at a pressure below 2×10⁻³ Pa to complete the device.

Eight different devices were manufactured. A control device did not include the first semiconducting layer (having phthalocyanine), and only used the second semiconducting layer. The other seven devices used H₂Pc, CuPc, ClAlPc, ClGaPc, ClInPc, TiOPc, and VOPc in the first semiconducting layer. Thus, the devices differed in the phthalocyanine used in the first semiconducting layer.

### Comparison

Devices were illuminated through the ITO electrode with 100 mW/cm² simulated sunlight using an Oriel 96000 solar simulator with an AM1.5G spectral filter. Input power was monitored with a Newport 818-UV/CM detector and Newport 1830-C optical power meter. A Keithley 238 source-measure unit and PC collected J-V data. The active device area was 7 mm² defined by a shadow mask.

External Quantum Efficiency (EQE) measurements were performed using a calibrated monochromator from Photon Technology International and a Kiethley 6485 picoammeter, measuring short circuit current as a function of incident wavelength. This property measures a device's electrical sensitivity to light, and provides information on the current that a given device will produce when illuminated by a particular wavelength.

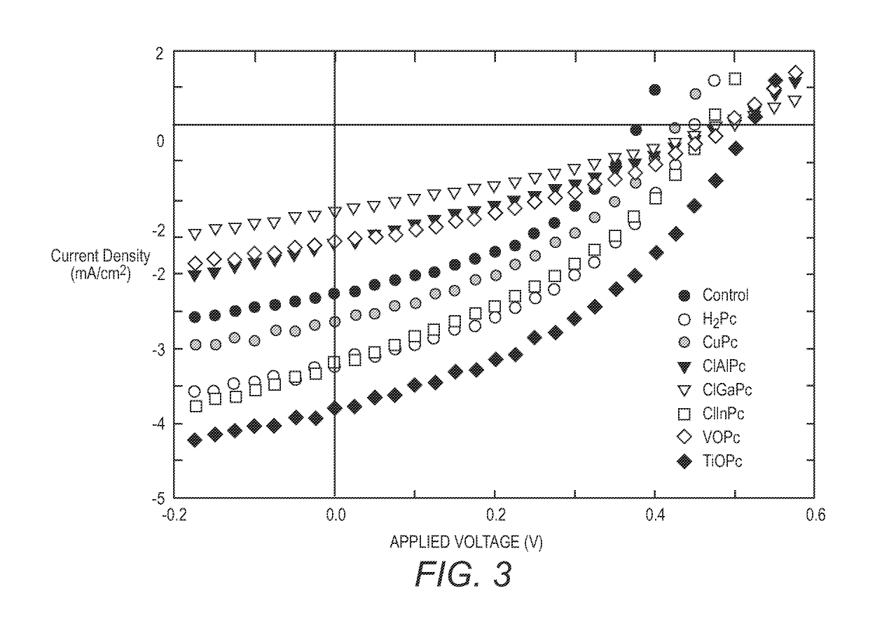

FIG. 3 is a graph showing the current density versus the applied voltage for the eight devices. Current density was calculated by dividing the current by the active area. Table 1 also summaries the results of testing the eight devices. The open circuit voltage V_{OC} is the voltage on the device when the current is zero. The short circuit current J_{SC} is the current flow when the voltage is zero. The fill factor FF is the ratio of the actual maximum obtainable power to the theoretical power. The power conversion efficiency PCE is the efficiency obtained by the device at its optimal load. Table 1 is sorted by increasing PCE.

TABLE 1

| Phthalocyanine | V_{OC} (V) | J_{SC} (mA/cm²) | FF (%) | PCE (%) |
|---|---|---|---|---|
| ClAlPc | 0.49 | 1.60 | 30 | 0.24% |
| ClGaPc | 0.50 | 1.17 | 31 | 0.30% |
| VOPc | 0.49 | 1.79 | 36 | 0.31% |
| Control | 0.38 | 2.26 | 43 | 0.37% |
| CuPc | 0.43 | 2.65 | 39 | 0.44% |
| ClInPc | 0.47 | 3.21 | 37 | 0.56% |
| H₂Pc | 0.45 | 3.22 | 42 | 0.61% |
| TiOPc | 0.52 | 3.80 | 40 | 0.79% |

For all devices containing phthalocyanine, the open circuit voltage V_{OC} was improved over the control device. The increase in V_{OC} can be attributed to improved energy level positioning, as the lower HOMO of the phthalocyanine increases the energy level difference from the LUMO of the PCBM. The highest V_{OC} was attained with the trivalent and tetravalent metallophthalocyanines.

The J_{SC} showed improvement in the CuPc, ClInPc, H₂Pc and TiOPc devices, compared to the control. Devices with improved J_{SC} had J-V curve shapes similar to the control, showing minimal decrease in fill factor. The three devices with worse J_{SC} compared to the control (ClAlPc, ClGaPc and VOPc) showed a lower slope at the V_{OC}. This is indicative of a higher series resistance causing reduced performance.

All seven phthalocyanine devices showed a reduced fill factor compared to the control. This reduction in fill factor can be explained by the additional series resistance and device thickness versus the control cell. The higher series resistance is likely due to the layer thickness being considerably greater than the exciton diffusion length in the seven phthalocyanine devices. Put another way, because the thickness of the first semiconducting layer was held constant, and since transport properties depend on the phthalocyanine, the seven devices were not necessarily optimized for each individual phthalocyanine. Improved performance could potentially be achieved by varying the film thickness of the phthalocyanine-containing semiconducting layer.

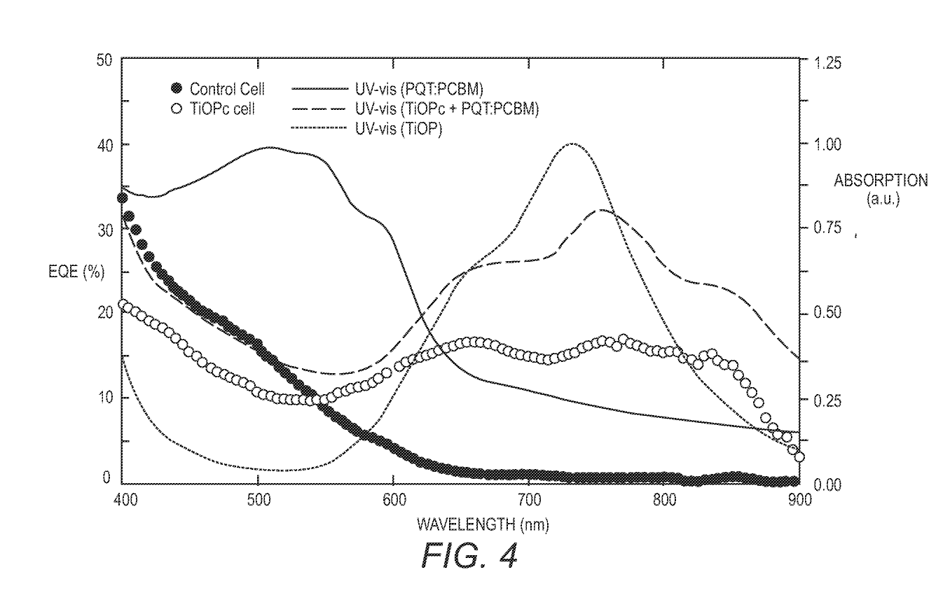

The external quantum efficiency (EQE) of the control device and the TiOPc device are shown in FIG. 4. The TiOPc device has a much higher EQE in the 550-850 nm range compared to the control device. The TiOPc device also continues generating current up to 900 nm. EQE values of 16% are reached within this range, compared to the minimal current of the control device. The additional current generated at the higher wavelengths explains the increase in J_{SC}. The optimal thickness for the first semiconducting layer for TiOPc was found to be between 16 and 20 nm, which is approximately three times the exciton diffusion length.

Also shown in FIG. 4 is the UV absorption profile of three different thin films. Film 1 is a PQT-12:PCBM film. Film 2 is a TiOPc film, which can be considered untreated. Film 3 contains a layer of TiOPc and a layer of PQT-12:PCBM. The TiOPc layer of Film 3 can be considered treated.

Initially, the absorption profiles for Film 1 and Film 2 are seen to be complementary. Film 2 (TiOPc) has a Q-band main peak at 730 nm, with a shoulder centered at 660 nm.

Film 3 shows a noticeable absorption red-shift and evolution of an IR shoulder. These changes can be attributed to a polymorph change during contact between 1,2-dichlorobenzene and TiOPc upon spinning of the upper PQT-12:PCBM layer. Put another way, the TiOPc in Film 2 is a different polymorph from the TiOPc in Film 3. Since the upper layer is spun directly onto the TiOPc layer, there is direct contact between solvent and TiOPc for the period of time between the dropping of the solution and commencement of spinning. This contact allows for relaxation and reorienting of the crystal structure within the TiOPc layer, i.e. a different polymorph. TiOPc films exposed solely to 1,2-dichlorobenzene solvent containing no PQT-12 or PCBM showed similar shifts in absorption spectra. Crystalline forms of TiOPc should have higher photosensitivity and improved transport properties due to a higher degree of molecular order. At the very least, in this study partial polymorph conversion appears to occur on the order of seconds during solvent exposure.

The present disclosure has been described with reference to exemplary embodiments. Obviously, modifications and alterations will occur to others upon reading and understanding the preceding detailed description. It is intended that the present disclosure be construed as including all such modifications and alterations insofar as they come within the scope of the appended claims or the equivalents thereof.

1. A photovoltaic device, comprising:
a substrate;
a first electrode upon the substrate;
a first semiconducting layer comprising a phthalocyanine;
a second semiconducting layer comprising a polythiophene and an electron acceptor; and
a second electrode;
wherein the first and second semiconducting layers are located between the first electrode and the second electrode, the first semiconducting layer being located closer in distance to the first electrode than the second semiconducting layer; and
wherein the polythiophene is of Formula (II): 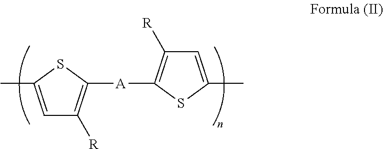
wherein A is a divalent linkage; each R is independently selected from hydrogen, alkyl, substituted alkyl, aryl, substituted aryl, alkoxy or substituted alkoxy, a heteroatom-containing group, halogen, —CN, or —NO₂; and n is from 2 to about 5,000.

2. The photovoltaic device of claim 1, wherein the phthalocyanine is of Formula (I): 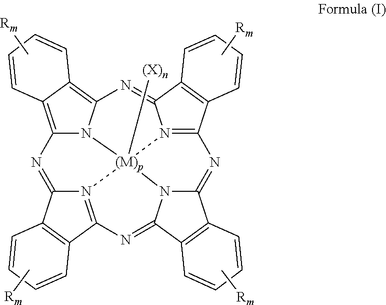
wherein M is a divalent, trivalent, or tetravalent metal atom; X is hydroxyl or halogen, and n is an integer from 0 to 2, or (X)ₙ is ═O; each m represents the number of R substituents on the phenyl ring, and is independently an integer from 0 to 6; each R is independently selected from the group consisting of halogen, alkyl, substituted alkyl, alkoxy, substituted alkoxy, phenoxy, phenylthio, aryl, substituted aryl, heteroaryl, —CN, and —NO₂; and p is 0 or 1.

3. The photovoltaic device of claim 1, wherein the phthalocyanine is titanium oxide phthalocyanine, indium chloride phthalocyanine, dihydrogen phthalocyanine, or copper phthalocyanine.

4. The photovoltaic device of claim 1, wherein the polythiophene is of Formula (III): 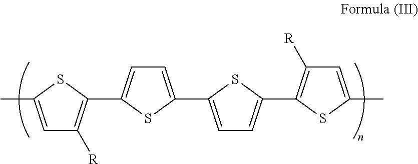
wherein R is alkyl.

5. The photovoltaic device of claim 1, wherein the polythiophene is of Formula (8):

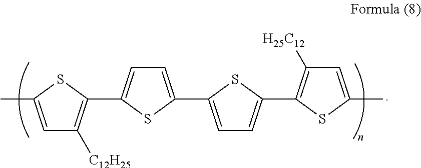

6. The photovoltaic device of claim 1, wherein the weight ratio of the polythiophene to the electron acceptor is from 1:99 to 99:1.

7. The photovoltaic device of claim 1, wherein the electron acceptor is C₆₀ fullerene, [6,6]-phenyl-C₆₁-butyric acid methyl ester (PCBM), C₇₀ fullerene, [6,6]-phenyl-C₇₁-butyric acid methyl ester, or a fullerene derivative.

8. The photovoltaic device of claim 1, wherein the first electrode comprises indium tin oxide, fluorine tin oxide, doped zinc oxide, poly(3,4-ethylenedioxythiophene):poly(styrene sulfonic acid) (PEDOT:PSS), carbon nanotube, or graphene.

9. The photovoltaic device of claim 1, wherein the second electrode comprises aluminum, silver, magnesium, calcium, or alloys thereof.

10. The photovoltaic device of claim 1, further comprising a hole blocking layer located between the second semiconducting layer and the second electrode.

11. The photovoltaic device of claim 10, wherein the hole blocking layer comprises bathocuproine, lithium fluoride, or bathophenanthroline.

12. The photovoltaic device of claim 1, further comprising an electron blocking layer between the first electrode and the first semiconducting layer.

13. The photovoltaic device of claim 12, wherein the electron blocking layer comprises poly(3,4-ethylenedioxythiophene):poly(styrene sulfonic acid) (PEDOT:PSS), MoO₃, or V₂O₅.

14. A photovoltaic device, comprising in sequence:
a substrate;
an anode upon the substrate;
an electron blocking layer;
a first semiconducting layer comprising a phthalocyanine;
a second semiconducting layer comprising a polythiophene and [6,6]-phenyl-C₆₁-butyric acid methyl ester (PCBM); and
a cathode upon the second semiconducting layer;
wherein the polythiophene is of Formula (II): 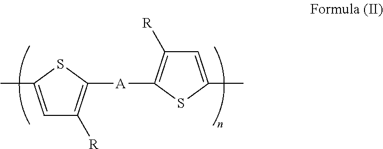
wherein A is a divalent linkage; each R is independently selected from hydrogen, alkyl, substituted alkyl, aryl, substituted aryl, alkoxy or substituted alkoxy, a heteroatom-containing group, halogen, —CN, or —NO₂; and n is from 2 to about 5,000.

15. The photovoltaic device of claim 15, wherein the phthalocyanine is of Formula (I): 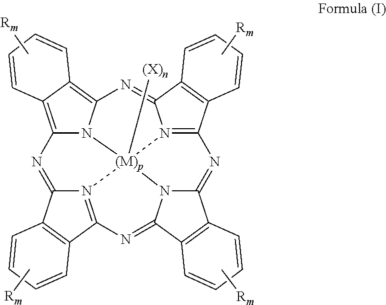
wherein M is a divalent, trivalent, or tetravalent metal atom; X is hydroxyl or halogen, and n is an integer from 0 to 2, or (X)ₙ is ═O; each m represents the number of R substituents on the phenyl ring, and is independently an integer from 0 to 6; each R is independently selected from the group consisting of halogen, alkyl, substituted alkyl, alkoxy, substituted alkoxy, phenoxy, phenylthio, aryl, substituted aryl, heteroaryl, —CN, and —NO₂; and p is 0 or 1.

16. The photovoltaic device of claim 16, wherein the polythiophene is of Formula (Ill): 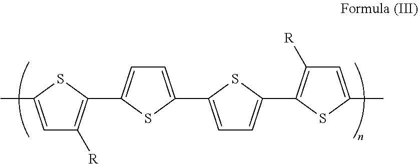
wherein R is alkyl.

17. The photovoltaic device of claim 14, wherein the polythiophene is of Formula (6):

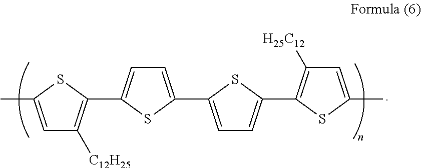

18. The photovoltaic device of claim 14, wherein the weight ratio of the polythiophene to PCBM is from 1:99 to 99:1.

19. The photovoltaic device of claim 1, further comprising a hole blocking layer located between the second semiconducting layer and the cathode.

20. A photovoltaic device, comprising in sequence:
an optically transparent substrate;
an indium tin oxide electrode upon the substrate;
an electron blocking layer comprising poly(3,4-ethylenedioxythiophene):poly(styrene sulfonic acid) (PEDOT:PSS);
a first semiconducting layer comprising oxytitanium phthalocyanine;
a second semiconducting layer comprising poly(3,3′″-didodecylquaterthiophene) and [6,6]-phenyl-C₆₁-butyric acid methyl ester (PCBM); and
an aluminum electrode deposited on the second semiconducting layer.
